(19) United States
(12) Patent Application Publication
(10) Pub. No.: US 2025/0199626 A1
(43) Pub. Date: Jun. 19, 2025

(54) SYSTEM AND METHOD FOR DETERMINING MEDICAL CODES

(71) Applicants: Heidi WALKER, Henderson, NV (US); Volodymyr HLUKHOVSKYI, Meridian, ID (US)

(72) Inventors: Heidi WALKER, Henderson, NV (US); Volodymyr HLUKHOVSKYI, Meridian, ID (US)

(21) Appl. No.: 18/986,570

(22) Filed: Dec. 18, 2024

Related U.S. Application Data
(60) Provisional application No. 63/611,750, filed on Dec. 18, 2023.

(51) Int. Cl. G06F 3/023; G16H 10/00

(52) U.S. Cl. CPC G06F 3/023; G16H 10/00

(57) ABSTRACT

Disclosed is a method, system, and non-transitory computer readable medium for determining medical codes

### CROSS REFERENCE TO RELATED APPLICATION

This application claims priority under 35 USC 119 from U.S. Provisional Application Ser. No. 63/611,750 filed on Dec. 18, 2023 and titled SYSTEM AND METHOD FOR DETERMINING MEDICAL CODES by Heidi Walker, et al., the entire disclosure of which is incorporated herein by reference.

### FIELD OF THE INVENTION

The present technology is in the field of computer systems and, more specifically, related to determining medical codes.

### BACKGROUND

Determining medical codes by a medical provider is generally a time consuming and error prone process. Typically, a medical billing specialist will review the medical notes regarding a patient, and then compare the medical notes to medical coding guidelines, such as International Classification of Diseases, to determine the appropriate medical codes.

### SUMMARY

The invention discloses a system, a method, and a non-transitory computer readable medium for determining medical codes.

### BRIEF DESCRIPTION OF THE DRAWINGS

In order to more fully understand the invention, reference is made to the accompanying drawings or figures. The invention is described in accordance with the aspects and embodiments in the following description with reference to the drawings or figures (FIG.), in which like numbers represent the same or similar elements. Understanding that these drawings are not to be considered limitations in the scope of the invention, the presently described aspects and embodiments and the presently understood best mode of the invention are described with additional detail through use of the accompanying drawings.

FIG. 1 shows a system having an identification of relevant medical codes process in accordance with the various aspects and embodiments of the invention.

FIG. 2 shows an identification of relevant medical codes process in accordance with the various aspects and embodiments of the invention.

FIG. 3 shows an identification of relevant medical codes process in accordance with the various aspects and embodiments of the invention.

FIG. 4 shows identifying relevant medical codes in accordance with the various aspects and embodiments of the invention.

### DETAILED DESCRIPTION

The following describes various examples of the present technology that illustrate various aspects and embodiments of the invention. Generally, examples can use the described aspects in any combination. All statements herein reciting principles, aspects, and embodiments as well as specific examples thereof, are intended to encompass both structural and functional equivalents thereof. Additionally, it is intended that such equivalents include both currently known equivalents and equivalents developed in the future, i.e., any elements developed that perform the same function, regardless of structure.

It is noted that, as used herein, the singular forms “a,” “an” and “the” include plural referents unless the context clearly dictates otherwise. Reference throughout this specification to “one aspect,” “an aspect,” “certain aspects,” “various aspects,” or similar language means that a particular aspect, feature, structure, or characteristic described in connection with any embodiment is included in at least one embodiment of the invention.

Appearances of the phrases “in one embodiment,” “in at least one embodiment,” “in an embodiment,” “in certain embodiments,” and similar language throughout this specification may, but do not necessarily, all refer to the same embodiment or similar embodiments. Furthermore, aspects and embodiments of the invention described herein are merely exemplary, and should not be construed as limiting of the scope or spirit of the invention as appreciated by those of ordinary skill in the art. The disclosed invention is effectively made or used in any embodiment that includes any novel aspect described herein. All statements herein reciting aspects and embodiments of the invention are intended to encompass both structural and functional equivalents thereof. It is intended that such equivalents include both currently known equivalents and equivalents developed in the future.

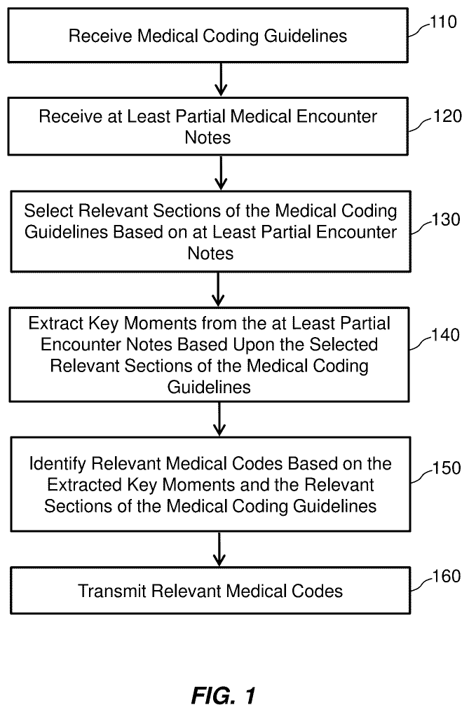

Referring now to FIG. 1, an identification of relevant medical codes process is shown, according to one or more embodiments and aspects of the invention. At step 110, medical coding guidelines may be received. Medical coding guidelines may include guidance to map descriptions of medical diagnoses or procedures into one or more medical codes. For example, medical coding guidelines may include ICD (International Classification of Diseases), International Classification of Functioning (ICF), Current Procedural Terminology (CPT), Healthcare Common Procedure Coding System (HCPCS), International Classification of Health Interventions (ICHI), and any other type of guidance to map descriptions of medical diagnoses or procedures into one or more medical codes. Medical coding guidelines may include specific versions of the guidelines, such as, ICD-11, ICD-10-CM, ICD-10-PCS, HCPCS Level II and any other specific version of the medical coding guidelines. Medical codes may include a representation of medical diagnosis, medical procedure, medical service, medical equipment, pharmaceutical drug, and any other medical code related to the medical diagnosis and/or treatment of a patient. Medical codes may include diagnostic codes, treatment codes, treatment modifier codes, equipment codes, pharmaceutical codes, topographical codes, and any other medical code related to the medical diagnosis and/or treatment of a patient. One or more medical coding guidelines may be received. The one or more received guidelines may be vectorized. The one or more medical coding guidelines may be stored in a database, such as, a vectorized database.

At step 120, at least partial medical encounter notes may be received. The medical encounter notes may include healthcare provider notes regarding a diagnosis and/or treatment of a patient. For example, the medical encounter notes may include a medical practitioner's, such as a physician, notes for a diagnosis and treatment of a patient disease. Partial medical notes may be received. For example, if a medical practitioner determines multiple diagnoses and performs multiple treatments, the encounter notes for a single diagnosis and the corresponding treatment may be received. Additionally, the entire medical encounter notes may be received.

At step 130, one or more relevant sections of the medical coding guidelines may be selected based on the at least partial medical encounter notes. The one or more relevant sections of the medical coding guidelines may be based upon the similarity with the at least partial medical encounter notes. For example, the one or more relevant sections of the medical coding guidelines may be based upon a similarity search between the at least partial medical encounter notes and the medical coding guidelines. The one or more relevant sections of the medical coding guidelines may be based upon context similarity with the at least partial medical encounter notes. The number of relevant sections of the medical coding guidelines and the size of the relevant sections of the medical coding guidelines may be predetermined. For example, two relevant sections of the medical coding guidelines may be selected with each section being one thousand tokens in length. One or more machine learning and/or artificial intelligence models may be used to select one or more relevant sections of the medical coding guidelines. In an example, a neural network may be trained to select relevant sections of the one or more medical coding guidelines, and the trained neural network may be used to select one or more relevant sections of the medical coding guidelines based upon the similarity with the at least partial medical encounter notes.

At step 140, key moments of the at least partial medical encounter notes may be extracted based upon the selected relevant sections of the medical coding guidelines. The key moments may include a summary of the at least partial medical encounter notes. When extracting key moments, filler words may be removed. One or more machine learning and/or artificial intelligence models may be used to extract key moments. A large language model (LLM) may be prompted to extract the key moments. For example, the LLM may be given a prompt that includes the at least partial medical encounter notes along with a request to determine the key moments from the at least partial medical encounter notes. For another example, the LLM may be given the prompt “give me key moments from medical notes which will be needed to determine CPT and ICD10-CM codes. Key moments will be matching with a table which contains CPT and ICD10-CM codes with description” along with a designation of medical notes followed by the at least partial medical encounter notes.

At step 150, relevant medical codes may be identified based on the extracted key moments and the relevant sections of the medical coding guidelines. The relevant medical codes may be based upon the similarity between the extracted key moments and the relevant sections of the medical coding guidelines. For example, relevant medical codes may be based upon a similarity between the extracted key moments and the relevant sections of the medical coding guidelines. Zero or more relevant medical codes may be identified. For example, relevant medical codes may include 2 CPT codes, 5 modifiers, and 2 ICD codes. One or more machine learning and/or artificial intelligence models may be used to identify relevant medical codes. In an example, a neural network may be trained to identify relevant medical codes, and the trained neural network may be used to identify relevant medical codes. The rationale for identifying the relevant medical codes may be included with the relevant medical codes. For example, the one or more relevant sections of the medical coding guidelines may be included with the relevant medical codes. For another example, key moments of the at least partial medical encounter notes may be included with the relevant medical codes.

At step 160, the relevant medical codes may be transmitted.

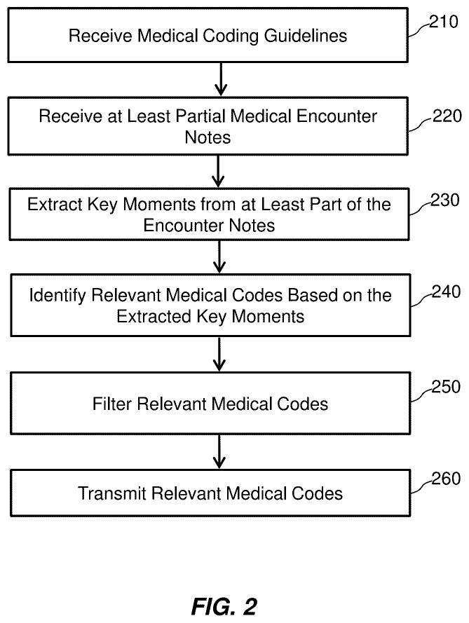

Referring now to FIG. 2, an identification of relevant medical codes process is shown, according to one or more embodiments and aspects of the invention. At step 210, medical coding guidelines may be received. In one or more examples, step 210 may be the same or similar to step 110. At step 220, at least partial medical encounter notes may be received. In one or more examples, step 220 may be the same or similar to step 120.

At step 230, key moments of the at least partial medical encounter notes may be extracted. The key moments may include a summary of the at least partial medical encounter notes. When extracting key moments, filler words may be removed. One or more machine learning and/or artificial intelligence models may be used to extract key moments. A large language model (LLM) may be prompted to extract the key moments. For example, the LLM may be given a prompt that includes the at least partial medical encounter notes along with a request to determine the key moments from the at least partial medical encounter notes. For another example, the LLM may be given the prompt “give me key moments from medical notes which will be needed to determine CPT and ICD10-CM codes. Key moments will be matching with a table which contains CPT and ICD10-CM codes with description” along with a designation of medical notes followed by the at least partial medical encounter notes.

At step 240, relevant medical codes may be identified based on the extracted key moments. The relevant medical codes may be based upon the similarity between the extracted key moments and the relevant sections of the medical coding guidelines. For example, relevant medical codes may be based upon a similarity between the extracted key moments and the relevant sections of the medical coding guidelines. Zero or more relevant medical codes may be identified. For example, relevant medical codes may include 2 CPT codes, 5 modifiers, and 2 ICD codes. One or more machine learning and/or artificial intelligence models may be used to identify relevant medical codes. In an example, a neural network may be trained to identify relevant medical codes, and the trained neural network may be used to identify relevant medical codes. The rationale for identifying the relevant medical codes may be included with the relevant medical codes. For example, the one or more relevant sections of the medical coding guidelines may be included with the relevant medical codes. For another example, key moments of the at least partial medical encounter notes may be included with the relevant medical codes.

At step 250, the relevant medical codes may be filtered. For example, the relevant medical codes may be filtered so that only the most relevant codes remain.

At step 260, the relevant medical codes may be transmitted. In one or more examples, step 260 may be the same or similar to step 160.

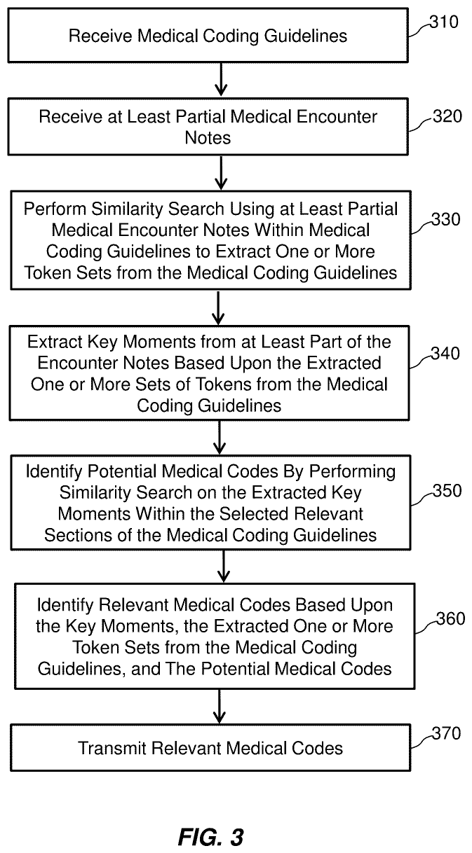

Referring now to FIG. 3, an identification of relevant medical codes process is shown, according to one or more embodiments and aspects of the invention. At step 310, medical coding guidelines may be received. In one or more examples, step 310 may be the same or similar to step 110. At step 320, at least partial medical encounter notes may be received. In one or more examples, step 320 may be the same or similar to step 120.

At step 330, a similarity search may be performed using the at least partial medical encounter notes within the medical coding guidelines to extract one or more token sets. Each token set may include a predetermined amount of tokens. For example, the one or more token sets may be based upon a similarity search between the at least partial medical encounter notes and the medical coding guidelines. The one or more token sets may be based upon context similarity with the at least partial medical encounter notes.

At step 340, key moments may be extracted from the at least partial of the encounter notes based upon the extracted one or more sets of tokens from the medical coding guidelines. The key moments may include a summary of the at least partial medical encounter notes. When extracting key moments, filler words may be removed. One or more machine learning and/or artificial intelligence models may be used to extract key moments. A large language model (LLM) may be prompted to extract the key moments. For example, the LLM may be given a prompt that includes the at least partial medical encounter notes along a request to determine the key moments from the at least partial medical encounter notes. For another example, the LLM may be given the prompt “give me key moments from medical notes which will be needed to determine CPT and ICD10-CM codes. Key moments will be matching with a table which contains CPT and ICD10-CM codes with description” along with a designation of medical notes followed by the at least partial medical encounter notes.

At step 350, potential medical codes may be identified by performing a similarity search on the extracted key moments within the selected relevant section of the medical coding guidelines. The relevant medical codes may be based upon the similarity between the extracted key moments and the relevant sections of the medical coding guidelines. For example, relevant medical codes may be based upon a similarity between the extracted key moments and the relevant sections of the medical coding guidelines. Zero or more relevant medical codes may be identified. For example, relevant medical codes may include 2 CPT codes, 5 modifiers, and 2 ICD codes. One or more machine learning and/or artificial intelligence models may be used to identify relevant medical codes. In an example, a neural network may be trained to identify relevant medical codes, and the trained neural network may be used to identify relevant medical codes. The rationale for identifying the relevant medical codes may be included with the relevant medical codes. For example, the one or more relevant sections of the medical coding guidelines may be included with the relevant medical codes. For another example, key moments of the at least partial medical encounter notes may be included with the relevant medical codes.

At step 360, relevant medical codes may be identified based upon the key moments, the extracted one or more token set from the medical coding guidelines, and the potential medical codes.

At step 370, the relevant medical codes may be transmitted. In one or more examples, step 370 may be the same or similar to step 160.

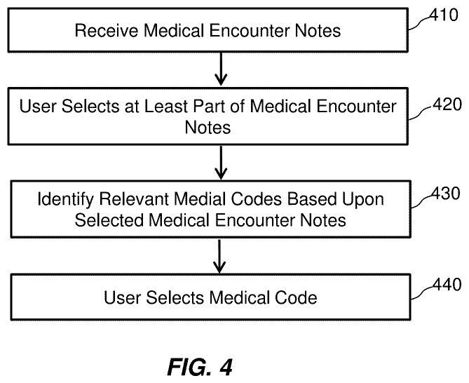

Referring now to FIG. 4, identifying relevant medical codes is shown, according to one or more embodiments and aspects of the invention. At step 410, medical encounter notes may be received. The medical encounter notes may include healthcare provider notes regarding a diagnosis and/or treatment of a patient. For example, the medical encounter notes may include a medical practitioner's, such as a physician, notes for a diagnosis and treatment of a patient disease. Partial medical notes may be received. For example, if a medical practitioner determines multiple diagnoses and performs multiple treatments, the encounter notes for a single diagnosis and the corresponding treatment may be received. Additionally, the entire medical encounter notes may be received.

At step 420 a user may select at least part of the medical encounter notes. In an example, a single diagnosis and the corresponding treatment is selected. In another example, the entire encounter notes may be selected. The user may select the part of the encounter notes that do not contain protected health information (PHI). Additionally, when the user is only allowed to select the encounter notes, the PHI may not be available as the encounter notes generally do not contain PHI.

At step 430, the relevant medical codes are identified based upon the selected medical encounter notes. The examples as taught by FIGS. 1-3 may be used to identify relevant medical codes. The relevant medical codes may be identified on a user device or another device. For example, the relevant medical codes may be identified on the user device. For another example, the relevant medical codes may be identified on a remote computer, such as, a remote server.

At step 440, the user may select the medical code from the relevant medical codes.

The above examples may provide numerous advantages. By formatting the prompts to a large AI model (e.g., large language model), the workload on the AI model may be reduced (e.g., less power used, less heat dissipated, etc.), the accuracy may be increased (e.g., improved result, less hallucinations, etc.), and the usage of the AI may be reduced (e.g., tokens used, faster results, less billed AI model usage, etc.). Also, the above examples may allow a human medical coder to more quickly determine medical codes and more accurately determine medical codes.

Certain examples have been described herein and it will be noted that different combinations of different components from different examples may be possible. Salient features are presented to better explain examples; however, it is clear that certain features may be added, modified, and/or omitted without modifying the functional aspects of these examples as described.

Practitioners skilled in the art will recognize many modifications and variations. The modifications and variations include any relevant combination of the disclosed features. Descriptions herein reciting principles, aspects, and embodiments encompass both structural and functional equivalents thereof. Elements described herein as “coupled” or “communicatively coupled” have an effectual relationship realizable by a direct connection or indirect connection, which uses one or more other intervening elements. Embodiments described herein as “communicating” or “in communication with” another device, module, or elements include any form of communication or link and include an effectual relationship. For example, a communication link may be established using a wired connection, wireless protocols, near-filed protocols, or RFID.

To the extent that the terms “including”, “includes”, “having”, “has”, “with”, or variants thereof are used in either the detailed description and the claims, such terms are intended to be inclusive in a similar manner to the term “comprising.”

The scope of the invention, therefore, is not intended to be limited to the exemplary embodiments and aspects that are shown and described herein. Rather, the scope and spirit of the invention is embodied by the appended claims.

What is claimed is:

1. A method for determining medical codes comprising: determining medical code.

2. A system including a processor and memory, wherein the memory stores code that is executed by the processor to cause the system to: determine medical code.

3. A non-transitory computer readable medium containing program instructions for causing a computer to perform the method of: determining medical codes.
